(19) United States
(12) Patent Application Publication
(10) Pub. No.: US 2010/0093866 A1
(43) Pub. Date: Apr. 15, 2010

(54) Pharmaceutical Formulations

(76) Inventors: Michael John Monteith, Stevenage (GB); Marian Thomas, Ware (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/641,394

(22) Filed: Dec. 18, 2009

Related U.S. Application Data
(62) Division of application No. 10/564,191, filed on May 22, 2006.
(60) Provisional application No. 60/505,415, filed on Sep. 23, 2003.

(30) Foreign Application Priority Data
Jul. 11, 2003 (GB) 0316341.7

(51) Int. Cl. A61K 31/18; A61K 47/14; A61K 47/26; A61K 31/135; A61P 43/00

(52) U.S. Cl. 514/603; 514/785; 514/777; 514/646

(57) ABSTRACT

The invention relates to the use of magnesium stearate to inhibit or reduce chemical degradation of an active ingredient substance in a formulation comprising a carrier in a solid pharmaceutical formulation, wherein the active ingredient substance is susceptible to chemical degradation.

The present invention relates to solid pharmaceutical formulations which comprise an active ingredient drug substance, a carrier and magnesium stearate. The invention also relates to the use of magnesium stearate to inhibit or reduce chemical reaction or degradation of an active ingredient substance in the presence of a carrier. The invention also relates to the use of magnesium stearate for the stabilisation of an active ingredient drug substance in the presence of a carrier.

An important requirement of pharmaceutical formulations is that they should be stable on storage in a range of different conditions. It is known that active ingredient substances can demonstrate instability to one or more of heat, light or moisture and various precautions must be taken in formulating and storing such substances to ensure that the pharmaceutical products remain in an acceptable condition for use over a reasonable period of time, such that they have an adequate shelf-life. Instability of a drug substance may also arise from contact with one or more other components present in a formulation, for example a component present as an excipient.

It is usual practice in the pharmaceutical art to formulate active ingredient substance with substances known as excipients which may be required as carriers, diluents, fillers, bulking agents, binders etc. Such excipients are often used to give bulk to a pharmaceutical formulation where the active ingredient substance is present in very small quantities. Such substances are generally chemically inert. Over prolonged storage times, or under conditions of extreme heat or humidity, and in the presence of other materials, such inert substances can, however, undergo or participate in chemical degradation reactions.

Carrier substances that are commonly utilised in solid pharmaceutical formulations include reducing sugars, for example lactose, maltose and glucose. Lactose is particularly commonly used. It is generally regarded as an inert excipient.

However, it has been observed that certain active ingredient substances may undergo a chemical reaction in the presence of lactose and other reducing sugars. For example, it was reported by Wirth et al. (J. Pharm. Sci., 1998, 87, 31-39) that fluoxetine hydrochloride (sold under the tradename Prozac®) undergoes degradation when present in solid tablets with a lactose excipient. The degradation was postulated to occur by formation of adducts via the Maillard reaction and a number of early Maillard reaction intermediates were identified. The authors conclude that drug substances which are secondary or primary amines undergo the Maillard reaction with lactose under pharmaceutically relevant conditions.

The present inventors have found that, under accelerated stability conditions, certain inhalable active ingredient substances also undergo degradation in the presence of lactose, possibly also via the Maillard reaction.

Some inhalable dry powder pharmaceuticals are sensitive to moisture, as reported, for example in WO 00/28979 (SkyePharma AG). The presence of moisture was found to interfere with the physical interaction between a carrier and a drug substance and thus with the effectiveness of drug delivery. Such interference with physical interactions between a carrier and a drug substance is distinct from chemical instability resulting from degradation.

A commonly used excipient in solid pharmaceutical formulations is magnesium stearate, which is often included as a lubricant. WO00/28979 (SkyePharma AG) describes the use of magnesium stearate in dry powder formulations for inhalation to improve resistance to moisture and to reduce the effect of penetrating moisture on the fine particle fraction (FPF) of an inhaled formulation. WO00/53158 (Chiesi) describes a powder for use in a dry powder inhaler including an active ingredient and a carrier, wherein the carrier includes a lubricant, which may, for example, be inter alia magnesium stearate.

WO 96/23485 (Coordinated Drug Development Ltd), WO01/78694 and WO01/78695 (Vectura Limited) each describes a powder for use in a dry powder inhaler including an active ingredient particles and carrier particles, wherein the carrier includes an additive which is able to promote release of the active particles from the carrier particles. Possible additive materials include amino acids, phospholipids, fatty acids and derivatives of fatty acids such as salts and esters, including inter alia magnesium stearate

We have now surprisingly found that chemical interaction of active ingredient substance and carrier may be inhibited or reduced by the presence of magnesium stearate.

In a first aspect therefore the present invention provides the use of magnesium stearate to inhibit or reduce chemical interaction between an active ingredient substance and a carrier in a solid pharmaceutical formulation, wherein said active ingredient substance is susceptible to chemical interaction with said carrier.

The invention also provides the use of magnesium stearate to inhibit or reduce chemical degradation of an active ingredient substance in a solid pharmaceutical formulation comprising the active ingredient substance and a carrier, wherein said active ingredient substance is susceptible to chemical interaction with said carrier. The chemical stability of the active substance in the formulation during long term storage may thereby be improved.

In a second aspect the present invention provides a solid pharmaceutical formulation comprising (a) an active ingredient substance susceptible to chemical interaction with a carrier, (b) a carrier and (c) magnesium stearate.

In a third aspect the present invention provides a method of reducing or inhibiting chemical interaction between an active ingredient substance and a carrier susceptible to chemical interaction, which comprises mixing magnesium stearate with said active ingredient substance and said carrier. The invention also provides a method of inhibiting chemical degradation of an active ingredient substance in a formulation comprising a carrier and an active ingredient substance, which method comprises mixing magnesium stearate with said active ingredient substance and said carrier.

Pharmaceutical formulations that have been prepared according to the present invention have greater chemical stability than the corresponding formulations without said ternary agent.

In the context of the present invention magnesium stearate may be referred to as a ternary agent. ‘Ternary agent’ is used herein to mean a compound used in a formulation in addition to the active ingredient drug substance or substances (the ‘primary’ agent) and a bulk carrier material or materials (the ‘secondary’ agent). In some circumstances more than one ternary agent may be used. Optionally, further substances, possibly named ‘quaternary agents’, may also be present, for example as a lubricant. Any particular ternary or quaternary agent may have more than one effect.

The invention finds particular application in formulations in which the carrier is a reducing sugar, for example lactose, maltose or glucose (for example monohydrate glucose or anhydrate glucose). In a preferred embodiment, the carrier is lactose. Alternative carriers include maltodextrin.

The optimal amount of magnesium stearate present in a particular composition varies depending on the identity of the active ingredient drug substance present, the sizes of the particles and various other factors. In general, magnesium stearate is preferably present in an amount of from 0.1 to 20% w/w based on the total weight of the composition. More preferably the magnesium stearate is present in an amount of from 0.2 to 10% w/w based on the total weight of the composition. Still more preferably, the magnesium stearate is present in an amount of from 0.3 to 6% w/w, for example from 0.5 to 4% w/w.

The active ingredient substance is typically present in an amount of from 0.01% to 50% w/w based on the total weight of the composition. Preferably, the active ingredient substance is present in an amount of from 0.02% to 10% w/w, more preferably in an amount of from 0.03 to 5% w/w, for example from 0.05% to 1% w/w, for example 0.1% w/w.

Preferably, the active ingredient drug substance is one which includes a primary or secondary amine group. Thus for example the drug substance may contain the group Ar—CH(OH)—CH₂—NH—R.

The group Ar may for example be selected from a group of formula (a) (b) (c) or (d):

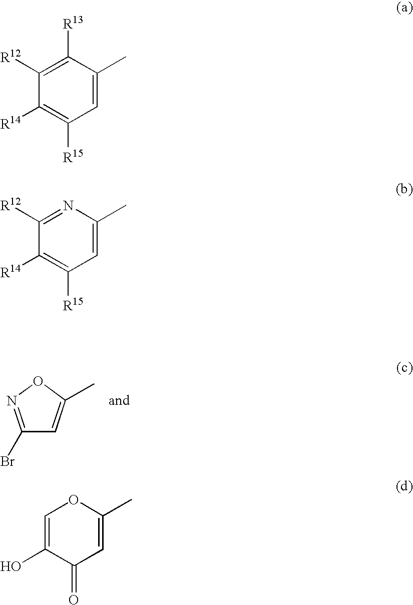

wherein R¹² represents hydrogen, halogen, —(CH₂)_{q}OR¹⁶, —NR¹⁶C(O)R¹⁷, —NR¹⁶SO₂R¹⁷, —SO₂NR¹⁶R¹⁷, —NR¹⁶R¹⁷, —OC(O)R¹⁸ or OC(O)NR¹⁶R¹⁷,

and R¹³ represents hydrogen, halogen or C₁₋₄ alkyl;

or R¹² represents —NHR¹⁹ and R¹³ and —NHR¹⁹ together form a 5- or 6-membered heterocyclic ring;

R¹⁴ represents hydrogen, halogen, —OR¹⁶ or —NR¹⁶R¹⁷;

R¹⁵ represents hydrogen, halogen, haloC₁₋₄ alkyl, —OR¹⁶, —NR¹⁶R¹⁷, —OC(O)R¹⁸ or OC(O)NR¹⁶R¹⁷;

R¹⁶ and R¹⁷ each independently represents hydrogen or C₁₋₄ alkyl, or in the groups —NR¹⁶R¹⁷, —SO₂NR¹⁶R¹⁷ and —OC(O)NR¹⁶R¹⁷, R¹⁶ and R¹⁷ independently represent hydrogen or C₁₋₄ alkyl or together with the nitrogen atom to which they are attached form a 5-, 6- or 7-membered nitrogen-containing ring,

R¹⁸ represents an aryl (eg phenyl or naphthyl) group which may be unsubstituted or substituted by one or more substituents selected from halogen, C₁₋₄ alkyl, hydroxy, C₁₋₄ alkoxy or halo C₁₋₄ alkyl; and

q is zero or an integer from 1 to 4.

In a particular embodiment, the group Ar is as defined above except that R¹² is not hydrogen.

Within the definitions of (a) and (b) above, preferred groups may be selected from the following groups (i) to (xxi):

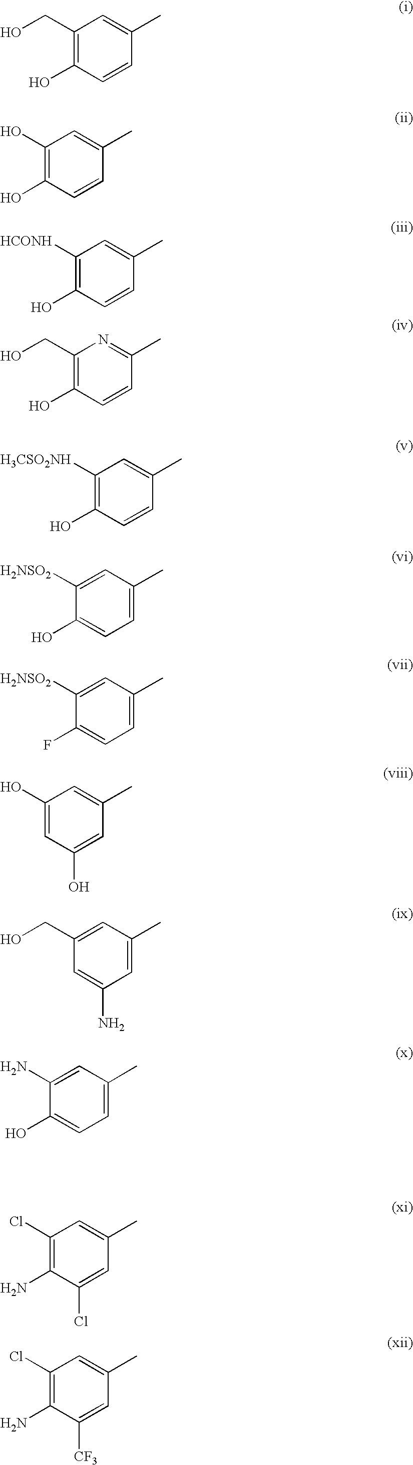

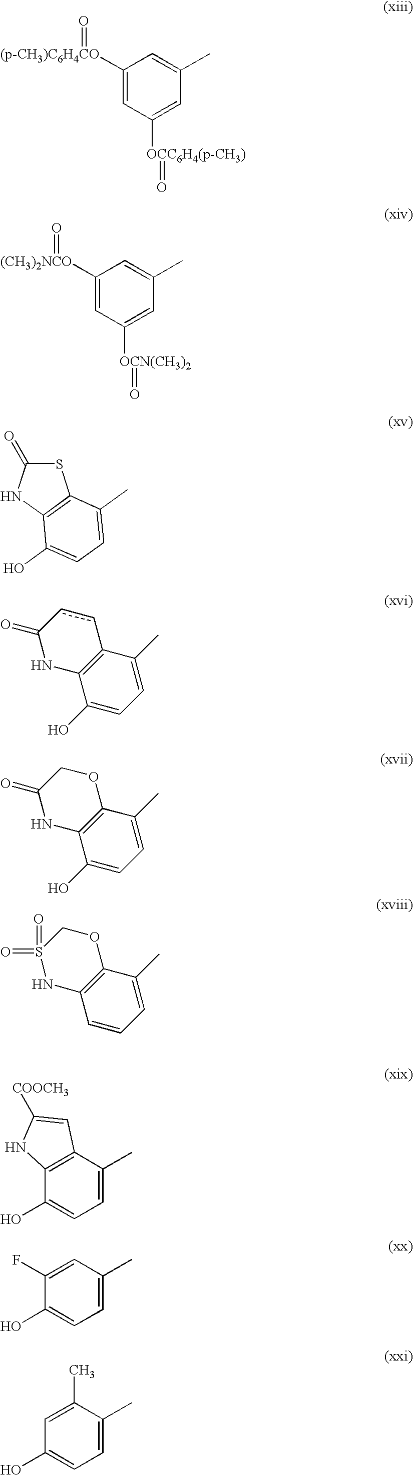

wherein the dotted line in (xvi) and (xix) denotes an optional double bond.

In a particular embodiment Ar represents a group (i) as defined above.

In another embodiment Ar represents a group (iii) as defined above.

The group R preferably represents a moiety of formula:

-A-B-C-D

wherein:

A may represent (CH₂)ₘ wherein m is an integer from 1 to 10;

B may represent a heteroatom, e.g. oxygen, or a bond;

C may represent (CH₂), wherein n is an integer from 1 to 10; and

D may represent an aryl group, e.g. an optionally substituted phenyl or pyridyl group.

Drug substances which may be formulated in accordance with the present invention include those described in International Patent Applications WO 02/066422, WO 02/070490, WO 02/076933, WO 03/024439, WO 03/072539, WO 03/091204, WO 04/016578, WO2004/022547, WO 2004/037807, WO 2004/037773, WO 2004/037768, WO 2004/039762, and WO 2004/039766.

Specific drug substances which may be formulated in accordance with the present invention include:

3-(4-{[6-({(2R)-2-hydroxy-2-[4-hydroxy-3-(hydroxymethyl)phenyl]ethyl}amino)hexyl]oxy}butyl)benzenesulfonamide for example as its cinnamate salt;

3-(3-{[7-({(2R)-2-hydroxy-2-[4-hydroxy-3-hydroxymethyl)phenyl]ethyl}-amino)heptyl]oxy}propyl)benzenesulfonamide;

4-{(1R)-2-[(6-{2-[(2,6-dichlorobenzyl)oxy]ethoxy}hexyl)amino]-1-hydroxyethyl}-2-(hydroxymethyl)phenol and

4-{(1R)-2-[(6-{4-[3-(cyclopentylsulfonyl)phenyl]butoxy}hexyl)amino]-1-hydroxyethyl}-2-(hydroxymethyl)phenol

and salts, solvates and other physiologically functional derivatives thereof.

Other drug substances which may be formulated in accordance with the present invention include salmeterol, (R)-salmeterol, salbutamol, (R)-salbutamol, formoterol, (R,R)-formoterol, fenoterol, etanterol, naminterol, clenbuterol, pirbuterol, flerobuterol, reproterol, bambuterol and terbutaline and salts, solvates and other physiologically functional derivatives thereof.

The active ingredient drug substance may be in the form of a free acid or base or may be present as a salt, a solvate, or other physiologically functional derivative. Salts and solvates which are suitable for use in medicine are those wherein the counterion or associated solvent is pharmaceutically acceptable.

Suitable salts for use in the invention include those formed with both organic and inorganic acids or bases. Pharmaceutically acceptable acid addition salts include those formed from hydrochloric, hydrobromic, sulphuric, citric, tartaric, phosphoric, lactic, pyruvic, acetic, trifluoroacetic, triphenylacetic, phenylacetic, substituted phenylacetic eg. methoxyphenylacetic, sulphamic, sulphanilic, succinic, oxalic, fumaric, maleic, malic, glutamic, aspartic, oxaloacetic, methanesulphonic, ethanesulphonic, arylsulphonic (for example p-toluenesulphonic, benzenesulphonic, naphthalenesulphonic or naphthalenedisulphonic), salicylic, glutaric, gluconic, tricarballylic, mandelic, cinnamic, substituted cinnamic (for example, methyl, methoxy, halo or phenyl substituted cinnamic, including 4-methyl and 4-methoxycinnamic acid and a-phenyl cinnamic acid (E or Z isomers or a mixture of the two)), ascorbic, oleic, naphthoic, hydroxynaphthoic (for example 1- or 3-hydroxy-2-naphthoic), naphthaleneacrylic (for example naphthalene-2-acrylic), benzoic, 4-methoxybenzoic, 2- or 4-hydroxybenzoic, 4-chlorobenzoic, 4-phenylbenzoic, benzeneacrylic (for example 1,4-benzenediacrylic) and isethionic acids. Pharmaceutically acceptable base salts include ammonium salts, alkali metal salts such as those of sodium and potassium, alkaline earth metal salts such as those of calcium and magnesium and salts with organic bases such as dicyclohexyl amine and N-methyl-D-glucamine.

A physiologically functional derivative of a drug substance may also be used in the invention. By the term “physiologically functional derivative” is meant a chemical derivative of a compound of having the same physiological function as the free compound, for example, by being convertible in the body thereto. According to the present invention, examples of physiologically functional derivatives include esters, for example compounds in which a hydroxyl group has been converted to a C₁₋₆alkyl, aryl, aryl C₁₋₆alkyl, or amino acid ester.

The active ingredient drug substance is most preferably a selective long-acting β₂-adrenoreceptor agonist. Such compounds have use in the prophylaxis and treatment of a variety of clinical conditions, including diseases associated with reversible airways obstruction such as asthma, chronic obstructive pulmonary diseases (COPD) (e.g. chronic and wheezy bronchitis, emphysema), respiratory tract infection and upper respiratory tract disease (e.g. rhinitis, including seasonal and allergic rhinitis).

Other conditions which may be treated include premature labour, depression, congestive heart failure, skin diseases (e.g. inflammatory, allergic, psoriatic, and proliferative skin diseases), conditions where lowering peptic acidity is desirable (e.g. peptic and gastric ulceration) and muscle wasting disease.

Formulations to which the present invention may be applied include those suitable for oral, parenteral (including subcutaneous, intradermal, intramuscular, intravenous and intraarticular), inhalation (including fine particle dusts or mists which may be generated by means of various types of metered dose pressurised aerosols, nebulisers or insufflators), rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing the active ingredient into association with the carrier and the magnesium stearate ternary agent as well as any other accessory ingredients. In general the formulations are prepared by uniformly and intimately bringing into association the active ingredient, lactose, magnesium stearate and any other accessory ingredients, and then, if necessary, shaping the product into the desired formulation.

Formulations of the present invention suitable for oral administration may be presented as discrete units such as capsules, cachets or tablets each containing a predetermined amount of the active ingredient; as a powder or granules. The active ingredient drug substance may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Formulations for parenteral administration include sterile powders, granules and tablets intended for dissolution immediately prior to administration. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerin or sucrose an acacia.

The invention finds particular application in dry powder compositions, in particular in dry powder compositions for topical delivery to the lung by inhalation.

Dry powder compositions for topical delivery to the lung by inhalation may, for example, be presented in capsules and cartridges of for example gelatine, or blisters of for example laminated aluminium foil, for use in an inhaler or insufflator. Packaging of the formulation may be suitable for unit dose or multi-dose delivery. In the case of multi-dose delivery, the formulation can be pre-metered (eg as in Diskus, see GB 2242134 or Diskhaler, see GB 2178965, 2129691 and 2169265) or metered in use (eg as in Turbuhaler, see EP 69715 or EP0237507). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing an active compound. Preferably, the strip is sufficiently flexible to be wound into a roll.

Medicaments for administration by inhalation desirably have a controlled particle size. The optimum particle size for inhalation into the bronchial system is usually 1-10 μm, preferably 2-5 μm (mass mean diameter, MMD). Particles having a size above 20 μm are generally too large when inhaled to reach the small airways. To achieve these particle sizes the particles of the active ingredient substance as produced may be size reduced by conventional means eg by micronisation. The desired fraction may be separated out by air classification or sieving. Preferably, the particles will be crystalline. In general, the particle size of the carrier, for example lactose, will be much greater than the drug substance within the present invention. It may also be desirable for other agents other than the active drug substance to have a larger particle size than the active drug substance. When the carrier is lactose it will typically be present as milled lactose, for example with a mass mean diameter (MMD) of 60-90 μm and with not more than 15% having a particle diameter of less than 15 μm.

The magnesium stearate will typically have a particle size in the range 1 to 50 μm, and more particularly 1-20 μm, e.g.1-10 μm.

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

The compounds and pharmaceutical formulations according to the invention may be used in combination with or include one or more other therapeutic agents, for example a beta-agonist may be used in combination with one or more other therapeutic agents selected from anti-inflammatory agents (for example a corticosteroid, or an NSAID,) anticholinergic agents (particularly an M₁, M₂, M₁/M₂ or M₃ receptor antagonist), other β₂-adrenoreceptor agonists, antiinfective agents (e.g. antibiotics, antivirals), or antihistamines.

Suitable corticosteroids include methyl prednisolone, prednisolone, dexamethasone, fluticasone propionate, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl)ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide, butixocort propionate, RPR-106541, and ST-126.

Suitable NSAIDs include sodium cromoglycate, nedocromil sodium, phosphodiesterase (PDE) inhibitors (e.g. theophylline, PDE4 inhibitors or mixed PDE3/PDE4 inhibitors), leukotriene antagonists, inhibitors of leukotriene synthesis, iNOS inhibitors, tryptase and elastase inhibitors, beta-2 integrin antagonists and adenosine receptor agonists or antagonists (e.g. adenosine 2a agonists), cytokine antagonists (e.g. chemokine antagonists) or inhibitors of cytokine synthesis.

Suitable anticholinergic agents are those compounds that act as antagonists at the muscarinic receptor, in particular those compounds which are antagonists of the M₁ and M₂ receptors. Exemplary compounds include the alkaloids of the belladonna plants as illustrated by the likes of atropine, scopolamine, homatropine, hyoscyamine; these compounds are normally administered as a salt, being tertiary amines.

Preferred anticholinergics include ipratropium (e.g. as the bromide), sold under the name Atrovent, oxitropium (e.g. as the bromide) and tiotropium (e.g. as the bromide) (CAS-139404-48-1).

Suitable antihistamines (also referred to as H₁-receptor antagonists) include any one or more of the numerous antagonists known which inhibit H₁-receptors, and are safe for human use. All are reversible, competitive inhibitors of the interaction of histamine with H₁-receptors. Examples of preferred anti-histamines include methapyrilene and loratadine.

The invention further provides the use of an inhalable solid pharmaceutical formulation according to the invention for the manufacture of a medicament for the treatment of diseases associated with reversible airways obstruction such as asthma, chronic obstructive pulmonary diseases (COPD) (e.g. chronic and wheezy bronchitis, emphysema), respiratory tract infection and upper respiratory tract disease (e.g. rhinitis, including seasonal and allergic rhinitis). The invention also provides a method for treating asthma, chronic obstructive pulmonary diseases (COPD), chronic or wheezy bronchitis, emphysema, respiratory tract infection upper respiratory tract, or rhinitis, including seasonal and allergic rhinitis comprising administering to a patient in need thereof an inhalable solid pharmaceutical formulation according to the invention.

In a further aspect, the invention provides a method of preparing a solid pharmaceutical preparation comprising combining in one or more steps: (a) an active ingredient substance susceptible to interaction with a carrier, (b) a carrier and (c) magnesium stearate.

### EXAMPLES

Test Compound

In the following examples, the drug compound, “Compound X” was the cinnamate salt of 3-(4-{[6-({(2R)-2-hydroxy-2-[4-hydroxy-3-(hydroxymethyl)phenyl]ethyl}amino)hexyl]oxy}-butyl)benzene-sulfonamide. The synthesis of compound X is described in Examples 45 and 46 in WO 02/066422.

Method

Preparation of Blends

Lactose monohydrate was obtained from Borculo Domo Ingredients as BP/USNF form. Before use, the Lactose Monohydrate was sieved through a coarse screen (mesh size 500 microns) to deaggregate the material. Compound X was micronised before use in an APTM microniser to give a MMD (mean mass diameter) of from 2 to 5 microns.

Magnesium stearate was obtained from Peter Greven with MMD<10 microns and used as supplied.

The magnesium stearate was combined with lactose monohydrate and blended using either a high shear mixer (a QMM, PMA or TRV series mixer) or a low shear tumbling blender (a Turbula mixer) to provide a ternary agent/drug premix, hereinafter referred to as blend A.

Final blend B was obtained by first pre-mixing an appropriate quantity of blend A with compound X and then blending that blend A/compound X premix with further blend A in a weight ratio appropriate to provide blend B containing the magnesium stearate in the required quantity, as indicated in Table 1 and Tables 2 and 3 below. The quantity of magnesium stearate in Tables 2 to 3 is the amount by weight of magnesium stearate present as a percentage of the total composition. The final concentration of compound X in the blends was 0.1% w/w calculated on the basis of the weight of free base drug present.

For use in example 2, the blended composition was transferred into blister strips or the type generally used for the supply of dry powder for inhalation and the blister strips were sealed in the customary fashion.

The quantity of the various materials used in the various blends are shown in Table 1:

TABLE 1

| | Mass of | Mass of | Mass of |
|---|---|---|---|
| Excipient | excipient | compound X | lactose |
| None | — | 0.14 g | 99.86 g |
| 2% Mg stearate | 2.00 g | 0.14 g | 97.86 g |
| 1% Mg stearate | 1.00 g | 0.14 g | 98.86 g |
| 0.5% Mg stearate | 0.50 g | 0.14 g | 99.36 g |

0.14 g of compound X in the form of the cinnamate salt was used to provide 0.1 g of compound X free base.

Decomposition Conditions

The blends prepared as described above were subjected to accelerated decomposition conditions in a controlled atmosphere stability cabinet. In the tables below, the conditions to which the blends were subjected are given with reference to the temperature and the % relative humidity, for example 30/60 is 30° C. and 60% relative humidity (RH). Samples were analysed for decomposition products after the time periods indicated in the tables.

Analysis of Purity of Blends after Subjection to Decomposition Conditions

LC analysis was conducted on a Supelcosil ABZ+PLUS column (150×4.6 mm ID), 3 micron, eluting with water containing 0.05% trifluoroacetic acid (solvent A) and acetonitrile containing 0.05% v/v trifluroacetic acid (solvent B), using the following elution gradient: time 0=90% solvent A, 10% solvent B; 40 mins=10% solvent A, 90% solvent B; 41-45 mins 90% solvent A, 10% solvent B,. Flow rate was 1 ml/min and the column temperature was 40° C. Detection was carried out by UV at 220 nm with a HP1100 series detector model G1314A-VWD. The area under the LC trace curve for the total impurities was compared with the total area under the curve, to give the % area/area figures given in Tables 2 and 3.

Results

### Example 1

### Comparison of Compound X/Lactose Blends Comprising Magnesium Stearate with Controls

TABLE 2

| | | Condition | Total Impurities |
|---|---|---|---|
| Blend Details | Timepoint | ° C./% RH | (% area/area) |
| Compound X with | Week 2 | 30/60 | 5.0 |
| Lactose only | | 40/75 | 8.9 |
| | MN6 | 30/60 | 12.7 |
| | | 40/75 | 17.4 |
| Compound X with | Week 2 | 30/60 | 3.4 |
| Lactose and 2% | | 40/75 | 5.3 |
| Magnesium Stearate | MN6 | 30/60 | 4.1 |
| | | 40/75 | 5.1 |

### Example 2

### Comparison of Compound X/Lactose Blends Comprising 0.5%, 1.0% and 2.0% Magnesium Stearate Filled into Blister Strips with Controls

TABLE 3

| | | Condition | Total Impurities |
|---|---|---|---|
| Blend Details | Timepoint | ° C./% RH | (% area/area) |
| Compound X with | Initial | Initial | 3.7 |
| Lactose only | MN1 | 25/60 | 3.7 |
| | | 30/60 | 4.3 |
| | | 40/75 | 6.3 |
| Compound X with | Initial | Initial | 3.2 |
| Lactose and 0.5% | MN1 | 25/60 | 3.0 |
| Magnesium Stearate | | 30/60 | 3.0 |
| | | 40/75 | 3.8 |
| Compound X with | Initial | Initial | 3.2 |
| Lactose and 1.0% | MN1 | 25/60 | 3.2 |
| Magnesium Stearate | | 30/60 | 3.3 |
| | | 40/75 | 3.8 |
| Compound X with | Initial | Initial | 3.1 |
| Lactose and 2.0% | MN1 | 25/60 | 3.2 |
| Magnesium Stearate | | 30/60 | 3.3 |
| | | 40/75 | 3.7 |

1.-14. (canceled)

15. A method of inhibiting chemical degradation of an active ingredient substance in a formulation comprising a carrier and an active ingredient substance, which method comprises mixing magnesium stearate with said active ingredient substance and said carrier.

16. A method as claimed in claim 15 wherein the carrier is a reducing sugar.

17.-19. (canceled)

20. A method as claimed in claim 16, wherein the carrier is lactose.

21. A method as claimed in claim 15, wherein the magnesium stearate is present in an amount of from 0.1 to 20% w/w based on the total weight of the composition.

22. A method as claimed in claim 15, wherein the active ingredient substance is present in an amount of from 0.01% to 50% w/w based on the total weight of the composition.

23. A method as claimed in claim 15, wherein said drug substance is selected from:
3-(4-{[6-({(2R)-2-hydroxy-2-[4-hydroxy-3-(hydroxymethyl)phenyl]ethyl}amino)hexyl]oxy}butyl)benzenesulfonamide;
3-(3-{[7-({(2R)-2-hydroxy-2-[4-hydroxy-3-hydroxymethyl)phenyl]ethyl}-amino)heptyl]oxy}propyl)benzenesulfonamide;
4-{(1R)-2-[(6-{2-[(2,6-dichlorobenzy]oxy]ethoxy}hexyl)amino]-1-hydrogethyl}-2-(hydroxymethyl)phenol and
4-{(1R)-2-[(6-{4-[3-(cyclopentylsulfonyl)phenyl]butoxy}hexyl)amino]-1-hydroxyethyl}-2-(hydroxymethyl)phenol,
or a pharmaceutically acceptable salt thereof.

24. A method of inhibiting chemical degradation of 4-{(1R)-2-[(6-{2-[(2,6-dichlorobenzyl)oxy]ethoxy}hexyl)amino]-1-hydroxyethyl}-2-(hydroxymethyl)phenol or a pharmaceutically acceptable salt thereof in a
formulation comprising a lactose carrier, said method comprises:
combining
a) said 4-{(1R)-2-[(6-{2-[(2,6-dichlorobenzy)oxy]ethoxy}hexyl)amino]-1-hydroxyethyl}-2-(hydroxymethyl)phenol or a pharmaceutically acceptable salt thereof
b) said lactose, and
c) magnesium stearate.

25. The method of claim 24, wherein the magnesium stearate is combined in an amount of from 0.1 to 20% w/w based on the total weight of the formulation.

26. The method of claim 24, wherein the 4-{(1 R)-2-[(6-{2-[(2,6-dichlorobenzyl)oxy]ethoxy}hexyl)amino]-1-hydroxyethyl}-2-(hydroxymethyl)phenol or a pharmaceutically acceptable salt thereof is combined in an amount of from 0.01% to 50% w/w based on the total weight of the formulation.
